(19) United States
(12) Patent Application Publication
(10) Pub. No.: US 2010/0166843 A1
(43) Pub. Date: Jul. 1, 2010

(54) PHARMACEUTICAL COMPOSITION COMPRISING A CAMPOTHECIN DERIVATIVE

(75) Inventors: Giancarlo Francese, Basel (CH); Jörg Ogorka, Steinen (DE); Jia-ai Zhang, Court Skillman, NJ (US)

Correspondence Address: LUCAS & MERCANTI, LLP, 475 PARK AVENUE SOUTH, 15TH FLOOR, NEW YORK, NY, 10016, US

(73) Assignee: SIGMA-TAU INDUSTRIE FARMACEUTICHE RIUNITE S.P.A., Roma (IT)

(21) Appl. No.: 12/525,012

(22) PCT Filed: Jan. 30, 2008
(86) PCT No.: PCT/US08/52384

§ 371 Date: Dec. 10, 2009

Related U.S. Application Data
(60) Provisional application No. 60/887,619, filed on Feb. 1, 2007.

(51) Int. Cl. A61K 31/4375; A61K 9/133; A61K 38/40; A61K 38/28; A61K 31/675; A61K 31/715; A61K 38/18; A61K 39/395; A61K 9/127; A61P 35/00

(52) U.S. Cl. 424/450; 514/283; 514/8; 514/4; 514/89; 514/61; 514/12; 424/130.1; 424/172.1

(57) ABSTRACT

The present invention relates to pharmaceutical compositions comprising a topoisomerase I inhibitor including, but not limited to, a camptothecin derivative.

### FIELD OF THE INVENTION

The present invention relates to pharmaceutical compositions comprising a topoisomerase I inhibitor, including but not limited to a camptothecin derivative.

### BACKGROUND OF THE INVENTION

Camptothecin derivatives are a class of compounds described in U.S. Pat. No. 6,242,457. Camptothecin derivatives, such as those disclosed in U.S. Pat. No. 6,242,457, present highly specific difficulties in relation to administration generally, including in particular problems of drug bioavailability because these derivatives have very poor water solubility.

7-t-Butoxyiminomethylcamptothecin is a quinoline-based alkaloid blocking, through a topoisomerase inhibition, cell division in cells that divide rapidly, such as cancer cells. The drug substance is very poorly soluble in aqueous media which hinders the delivery of the effective amount of drug to the cancer cells.

In addition, 7-t-butoxyiminomethylcamptothecin is susceptibly to hydrolysis, and at physiological pH (˜7.4) the lactone ring tends to open readily, resulting in drug inactivation. In blood plasma the lactone ring is quickly opened to create the carboxylate form of the drug, which is poorly accumulated in cancer cells. Once internalized by the cancer cells, the carboxylate form exhibits no activity against its molecular target, topoisomersase I. Thus, the hydrolysed product is ineffective at treating cancer. Therefore, there is a need to develop a drug formulation comprising camptothecin derivatives, including but not limited to 7-t-butoxyiminomethylcamptothecin, that is stable, able to deliver clinical relevant dose to the cancer cells and is easy to use.

### SUMMARY OF THE INVENTION

The present invention overcomes the instability and poor solubility problems of camptothecin derivatives, including 7-t-butoxyiminomethylcamptothecin, when administered in its free form, by forming an unique pharmaceutically active composition containing functionalized phospholipids. This stabilized formulation can be used for an iv and subcutaneous administration.

In another aspect, the invention is directed:
a) to stabilize and increase the circulating time of a camptothecin derivative, including, but not limited to, 7-t-butoxyiminomethylcamptothecin, in blood; and
b) to increase the drug anti-tumor efficacy and to improve the action on a wider range of cancer diseases, through a drug targeting strategy.

### DETAILED DESCRIPTION OF THE INVENTION

The active agent is an inhibitor of topoisomerase I (Topo I inhibitor) and is therefore capable of preventing disease symptoms that are caused inter alia by the activation of the topoisomerase I receptor.

The camptothecin derivatives of the present invention, which are described in U.S. Pat. No. 6,242,457 include:
7-methyoxyiminomethylcamptothecin;
7-methoxyiminomethyl-10-hydroxycamptothecin;
7-(tert-butoxycarbonyl-2-propoxy)iminomethylcamptothecin;
7-ethoxyiminomethylcamptothecin;
7-isopropoxyiminomethylcamptothecin;
7-(2-methylbutoxy)iminomethylcamptothecin;
7-t-butoxyiminomethylcamptothecin;
7-t-butoxyiminomethyl-10-hydroxycamptothecin;
7-t-butoxyiminomethyl-10-methoxycamptothecin;
7-(4-hydroxybutoxy)iminomethylcamptothecin;
7-triphenylmethoxyiminomethylcamptothecin;
7-carboxymethoxyiminomethylcamptothecin;
7-(2-amino)ethoxyiminomethylcamptothecin;
7-(2-N,N-dimethylamino)ethoxyiminomethylcamptothecin;
7-allyloxyiminomethylcamptothecin;
7-cyclohexyloxyiminoethylcamptothecin;
7-cyclohexylmethoxyiminomethylcamptothecin;
7-cyclooctyloxyiminomethylcamptothecin;
7-cyclooctylmethoxyiminomethylcamptothecin;
7-benzyloxyiminomethylcamptothecin;
7-[(1-benzyloxyimino)-2-phenylethyl] camptothecin;
7-(1-benzyloxyimino)ethylcamptothecin;
7-phenoxyiminomethylcamptothecin;
7-(1-t-butoxyimino)ethylcamptothecin;
7-p-nitrobenzyloxyiminomethylcamptothecin;
7-p-methylbenzyloxyiminomethylcamptothecin;
7-pentafluorobenzyloxyiminomethylcamptothecin;
7-p-phenylbenzyloxyiminomethylcamptothecin;
7-[2-(2,4-difluorophenyl)ethoxy]iminomethylcamptothecin;
7-(4-t-butylbenzyloxy)iminomethylcamptothecin;
7-(1-adamantyloxy)iminomethylcamptothecin;
7-(1-adamantylmethoxy)iminomethylcamptothecin;
7-(2-naphthyloxy)iminomethylcamptothecin;
7-(9-anthrylmethoxy)iminomethylcamptothecin;
7-oxiranylmethoxyiminomethylcamptothecin;
7-(6-uracyl)methoxyiminomethylcamptothecin;
7-[2-(1-urcyl)ethoxy]iminomethylcamptothecin;
7-(4-pyridyl)methoxyiminomethylcamptothecin;
7-(2-thienyl)methoxyiminomethylcamptothecin;
7-[(N-methyl)-4-piperidinyl]methoxyiminomethylcamptothecin;
7-[2-(4-morpholininyl]ethoxy]iminomethylcamptothecin;
7-(benzoyloxyiminomethyl) camptothecin;
7-[(1-hydroxyimino)-2-phenylethyl) camptothecin;
7-tert-butyloxyiminomethylcamptothecin-N-oxide; and
7-methoxyiminomethylcamptothecin N-oxide.

In a very preferred embodiment of the invention, the topoisomerase I inhibitor of formula (I) has the following structure known as Compound A:

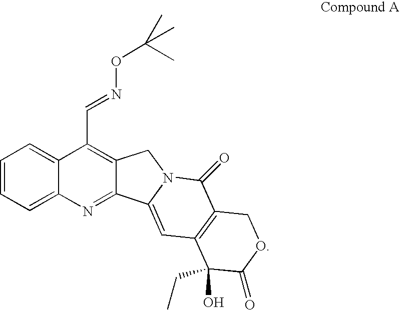

The preferred and especially preferred active agents, in free or pharmaceutically acceptable salt form, may be prepared as described in U.S. Pat. No. 6,424,457. As mentioned therein, they may be in the form of their possible enantiomers, diastereoisomers and relative mixtures, the pharmaceutically acceptable salts thereof and their active metabolites.

In one embodiment the present invention provides a pharmaceutical composition comprising:
a) a therapeutically effective amount of 7-t-butoxyiminomethylcamptothecin entrapped in liposomes; and
b) a pharmaceutically acceptable excipient.

The present invention provides a stable, highly pharmacologically active formulation by solubilizing the drug in phospholipids comprising 7-t-butoxyiminomethylcamptothecin. The formulation is in the form of liposomes, comprised of multiple phospholipids, such as conventional phospholipid, such as phosphatidylcholine cholesterol and the functionalized lipid. Typically, 7-t-butoxyiminomethylcamptothecin binds the lipid bilayer the membrane of liposome with high affinity. The 7-t-butoxyiminomethylcamptothecin intercalates between the acyl chains of the lipid, thereby reducing the lactone ring of the drug from interacting with the aqueous environment inside and outside the liposomes and thus protected from hydrolysis.

The liposome composition of the present invention is composed primarily of vesicle-forming lipids. Such a vesicle-forming lipid is one which:
a) can form spontaneously into bilayer vesicles in water, as exemplified by the phospholipids, or
b) is stably incorporated into lipid bilayers, with its hydrophobic moiety in contact with the interior, hydrophobic region of the bilayer membrane, and its head group moiety oriented toward the exterior and interior, polar surface of the vesicle.

The vesicle-forming lipids of this type are preferably ones having two hydrocarbon chains, typically acyl chains, and a head group, either polar or non-polar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids and sterols such as cholesterol or cholesterol derivatives.

Preferred diacyl-chain lipids for use in the present invention include diacyl glycerol, such as phosphatidylcholine (PC), phosphatidyl ethanolamine (PE), phosphatidylglycerol (PG), phosphatidylserine (PS), phosphatidic acid (PA), phosphatidylinositol (PI), sphingomyelin (SPM) and the like, alone or in combination.

The present invention overcomes the instability and poor solubility problems of 7-t-butoxyiminomethylcamptothecin when administered in its free form by forming an unique pharmaceutically active composition containing functionalized phospholipids. The functionalized phospholipids are those that surface grafted with certain hydrophilic polymers, and/or with certain ligands.

The surface drafted hydrophilic polymer is formed by including, at least in the outer lipid layer of the liposomes. Suitable hydrophilic polymers that are intended to extend liposome-circulation time, include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol and polyaspartamide.

In a preferred embodiment, the hydrophilic polymer is polyethyleneglycol, preferably as a PEG chain having a molecular weight between 500-10,000 daltons, typically between 1,000-5,000 daltons.

The surface grafted liposome provided by the hydrophilic polymer chains provides colloidal stability and serves to protect the liposomes from uptake by the reticulo-endothelial system, providing an extended blood circulation lifetime for the liposomes to reach the target cells. The extent of enhancement of blood circulation time is preferably several fold over that achieved in the absence of the polymer coating.

Examples of specific ligands for liposomes functionalization may include folic acid, peptides, proteins, enzymes, lectins, biotin, avidin, mono-, oligo-, and polysaccharides, hormones, cytokines, polyclonal and monoclonal antibodies including chimeric and humanized ones and their fragments.

In another embodiment the present invention provides a method of treating a cellular proliferative disease comprising administering to a mammalian host a pharmaceutical composition comprising:
a) a therapeutically effective amount of 7-t-butoxyiminomethylcamptothecin entrapped in liposomes; and
b) a pharmaceutically acceptable excipient
   The mammalian host may be a human.

In a further embodiment the present invention provides the use of a pharmaceutical composition of the present invention for the treatment of disease symptoms that are caused by the activation of the topoisomerase I receptor.

In a further embodiment the present invention provides use of the composition of the present invention for the preparation of a medicament for the treatment of disease symptoms that are caused by the activation of the topoisomerase I receptor.

The stabilized 7-t-butoxyiminomethylcamptothecin formulation circulates for prolonged period with better drug retention and plasma stability, leading to either passive or active preferential location into the tumor cells (as compared to a conventional formulation) through the Enhanced Permeation and Retention (EPR) effect and/or targeted delivery through specific cell surface receptors recognition. The stabilized 7-t-butoxyiminomethylcamptothecin formulation can be used for an iv and subcutaneous administration.

In a human of about 70 kg body weight, for example, from about 0.5-5 mg 7-t-butoxyiminomethylcamptothecin per kg of body weight can be administered. Preferably, about 1.0-3.0 mg of 7-t-butoxyiminomethylcamptothecin per kg of body weight is administered. However, it can be necessary to deviate from the dosages mentioned and in particular to do so as a function of the nature and body weight of the subject to be treated, the nature and the severity of the illness, the nature of the preparation and if the administration of the medicine, and the time or interval over which the administration takes place. Thus, it can suffice in some cases to manage with less that the above-mentioned amount of active compound whilst in other cases the above-mentioned amount of active compound must be exceeded. The particular required optimum dosage and the type of administration of 7-t-butoxyiminomethylcamptothecin can be determined by one skilled in the art, by available methods. Suitable amounts are therapeutically effective amounts that do not have excessive toxicity, as determined in empirical studies.

With the pharmaceutical compositions of the present invention, 7-t-butoxyiminomethylcamptothecin could be safely and effectively delivered by intravenous administration or into other body compartments.

The benefits of the present invention are that we could solve the problem of 7-t-butoxyiminomethylcamptothecin poor solubility and the low stability of the molecule in physiological pH intended to be used for iv and/or subcutaneous administration. Additional benefits are that with liposomes grafted with certain polymers we could increase the circulation time through the EPR effect and, through a functionalization of the liposomes/micelles with specific ligands, we could transport and enhance the cell internalization of 7-t-butoxyiminomethylcamptothecin to the targeted tumor cells more effectively compared to a conventional formulation.

7-t-Butoxyiminomethylcamptothecin can also be stabilized by entrapping in the hydrophobic region of micelles, and bound to the micelle membrane.

The present invention is directed:
a) to stabilize and increase the circulating time of the 7-t-butoxyiminomethylcamptothecin in blood; and
b) to increase the drug anti-tumor efficacy and to improve the action on a wider range of cancer diseases, through a drug targeting strategy.

### EXAMPLES

### Example 1

### 7-t-Butoxyiminomethylcamptothecin in Small Unilamellar, Long-Circulating Liposomes: Surface Grafted with Certain Polymers (Ex. Peg: Polyethylene Glycol).

### 7-t-Butoxyiminomethylcamptothecin in PEG-Liposome

The sample was prepared following the thin film hydration method also called Bangham method (Ref. Bangham A. D. & al., J. Mol. Biol. 13, 238-252, 1965) with the following adaptations:

STEP 1: preparation of the drug substance (DS), lipid film. Excipients and DS are dissolved in Ethanol. The organic solvent is evaporated off on a rotavapor (Rotavap R-210/215 from Büchi Switzerlans) for 4 hr at 40° C. to obtain a very homogenous DS, lipid film. The thin film obtained is maintained on rotavap for 2 hr, 55° C. and 30 mbar.
STEP 2: hydration of the DS, lipid film. To the DS, lipid film is added PB-Man buffer solution (pH 7.4) under magnetic stirring and at 40° C. for 30 min. A milky solution is obtained: the liposomal solution. The solution is put in an ultra-sound bath for 10 min at RT.
STEP 3: Freeze thawing of the liposomal solution. The liposomal solution is put in a liquid nitrogen (until solidification) and warmed in a 40° C. water bath (until melting) for 3 cycles.
STEP 4: Extrusion of the liposomal solution. The liposomal solution is extruded (LIPEX® Extruder from Norther Lipids Inc.) through polycarbonate filters (400 and 100 nm).
STEP 5: Sterilization. The liposomal solution is filtered on a sterile Millipore® filter 0.2 μm.

Sample composition

| | Concentration | |
|---|---|---|
| Ingredients | (mg/mL) | Volume (mL) |
| Phosphatidylcholine | 50 | — |
| MPEG-2000-DSPE | 13 | — |
| Cholesterol | 5 | — |
| D,L α-tocopherol | 0.25 | — |
| 7-t-Butoxyiminomethylcamptothecin | 0.15 | — |
| Phosphate buffer pH 5.4 | — | 30 |

Analytical characterization

| | |
|---|---|
| Analytical test | Results |

| Appearance (by visual examination) | Slightly green-yellow, | |
|---|---|---|
| | translucent dispersion | |
| pH-value | 5.44 | |
| HPLC identification of the DS | Positive | |
| HPLC assay | 0.11 | mg/mL |
| HPLC degradation products | Each ≦ 0.5%; sum ≦ 2.0% | |
| Mean particle size | 102 | nm |
| Particle size distribution | 99% < 282 | nm |
| Specific turbidity | 527.1 | NTU |

| | |
|---|---|
| Drug encapsulation | 96% |

### Example 2

### 7-t-Butoxyiminomethylcamptothecin in Liposomes with Ligands: Surface Grafted with Certain Ligands for a Drug Targeting Strategy

### 7-t-Butoxyiminomethylcamptothecin in PEG-Liposome

The sample was prepared following the thin film hydration method as described in example 1.

Sample composition

| | Concentration | |
|---|---|---|
| Ingredients | (mg/mL) | Volume (mL) |
| Phosphatidylcholine | 100 | — |
| MPEG-2000-DSPE | 26 | — |
| Cholesterol | 10 | — |
| D,L α-tocopherol | 0.5 | — |
| 7-t-Butoxyiminomethylcamptothecin | 0.25 | — |
| Phosphate buffer pH 5.4 | — | 10 |

Analytical characterization

| Analytical test | Results | |
|---|---|---|
| Appearance (by visual examination) | Slightly green-yellow, | |
| | translucent dispersion | |
| Mean particle size | 122 | nm |
| Particle size distribution | 99% < 402 | nm |

Stability test at 5° C. and 25° C.

| | Mean particle size | Mean particle size |
|---|---|---|
| Time (weeks) | (nm) at 5° C. | (nm) at 25° C. |
| 0 | 122 | 122 |
| 1 | 125 | 124 |
| 2 | 127 | 128 |
| 4 | 127 | 127 |
| 8 | 124 | 126 |

Plasma stability test at 37° C.

| | Mean particle size | Mean particle size |
|---|---|---|
| Time (hrs) | (nm) in 50% plasma | (nm) in 70% plasma |
| 0 | 129 | 131 |
| 0.75 | 123 | 121 |
| 1.5 | 120 | 118 |
| 3 | 119 | 118 |
| 6 | 120 | 119 |
| 24 | 126 | 121 |

### Example 3

### 7-t-Butoxyiminomethylcamptothecin in Long-Circulating Phospholipids Micelles: Surface Grafted with Certain Polymers (Ex. PEG2000: Polyethylene Glycol)

### 7-t-Butoxyiminomethylcamptothecin in PEG-Liposome

The sample was prepared following the thin film hydration method as described in the example 1. The only difference is in the STEP 4, the extrusion of the liposomal solution through polycarbonate filters (100 and 50 nm).

Sample composition

| | Concentration | |
|---|---|---|
| Ingredients | (mg/mL) | Volume (mL) |
| Phosphatidylcholine | 100 | — |
| MPEG-2000-DSPE | 26 | — |
| Cholesterol | 10 | — |
| D,L α-tocopherol | 0.5 | — |
| 7-t-Butoxyiminomethylcamptothecin | 0.25 | — |
| Phosphate buffer pH 5.4 | — | 40 |

Analytical characterization

| Analytical test | Results | |
|---|---|---|
| Appearance (by visual examination) | Slightly green-yellow, | |
| | translucent dispersion | |
| pH-value | 5.40 | |
| Mean particle size | 108 | nm |
| Particle size distribution | 99% < 293 | nm |
| Osmolarity | 362 | mOsm |
| Specific turbidity | 538.9 | NTU |
| Residual ethanol | <0.98% | (m/m) |

Stability test at 5° C. and 25° C.

| | Mean particle size | Mean particle size |
|---|---|---|
| Time (weeks) | (nm) at 5° C. | (nm) at 25° C. |
| 0 | 108 | 108 |
| 1 | 105 | 106 |
| 2 | 103 | 103 |
| 4 | 104 | 104 |

### Example 4

### 7-t-Butoxyiminomethylcamptothecin in Long-Circulating Phospholipids Micelles: Surface Grafted with Certain Polymers (Ex. PEG2000: Polyethylene Glycol) and with Certain Ligands (e.g., Folic Acid)

### 7-t-Butoxyiminomethylcamptothecin in Folic Acid Functionalized PEG-Liposome

The sample was prepared following the thin film hydration method as described in the example 1.

Sample composition

| | Concentration | |
|---|---|---|
| Ingredients | (mg/mL) | Volume (mL) |
| Phosphatidylcholine | 50 | — |
| MPEG-2000-DSPE | 12.5 | — |
| Folic acid funct. PEG-DSPE | 0.5 | — |
| Cholesterol | 5 | — |
| D,L α-tocopherol | 0.25 | — |
| 7-t-Butoxyiminomethylcamptothecin | 0.15 | — |
| Phosphate buffer pH 5.4 | — | 10 |

1. A method of treating a cellular proliferative disease comprising administering to a mammalian host a pharmaceutical composition comprising:
a) a therapeutically effective amount of 7-t-butoxyiminomethylcamptothecin entrapped in liposomes; and
b) a pharmaceutically acceptable excipient.

2. The method of claim 1, wherein said mammalian host is a human.

3. The method of claim 1, wherein the liposomes are made from synthetic or natural phospholipids that are selected from the group consisting of phosphatidyicholine, distearoylphosphatidylcholine, sphingomyelin, diacyl glycerol, phosphatidyl ethanolamine, phosphatidylglycerol, distearylphosphatidylcholine and distearyl phosphatidylethanolamine.

4. The method of claim 1, wherein the liposomes have a negative charge.

5. The method of claim 1, wherein the liposomes are neutral.

6. The method of claim 1, wherein the liposomes have a positive charge.

7. The method of claim 1, wherein the liposome surfaces are grafted with a hydrophilic polymer.

8. The method of claim 7, wherein said hydrophilic polymer is composed of hydrophilic polymers selected from the group consisting of polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethyl cellulose, polyethyleneglycol and polyaspartamide.

9. The method of claim 8, wherein said hydrophilic polymer coating is composed of polyethylene glycol chains having a molecular weight of between about 500 Daltons and about 10,000 Daltons.

10. The method of claim 1, wherein the liposomes are about 0.05 to about 1 microns.

11. The method of claim 1, wherein said liposomes further contain a ligand attached to the distal end of at least a portion of said hydrophilic polymer chains.

12. The method of claim 1, wherein the liposomes further include a ligand attached the polar head group of at least a portion of the vesicle-forming lipids of the liposome.

13. The method of claim 11, wherein the ligand is selected from the group consisting of folic acid, pyridoxal phosphate, sialyl Lewis, transferrin, epidermal growth factor, basic fibroblast growth factor, vascular endothelial growth factor, VCAM-1, ICAM-1, PECAN-1 and RGD peptides.

14. The method of claim 11, wherein the ligand is selected from the group consisting of water soluble vitamins, apolipoproteins, insulin, galactose, Mac-1, PECAM-1/CD31, fibronectin, osteopontin, RGD sequences of matrix proteins, HIV GP 120/41 domain peptomers, GP120C4 domain peptomers, T cell tropic isolates, SDF-1 chemokines, Macrophage tropic isolates, anti-cell surface receptor antibodies or fragments thereof, pyridoxyl ligands, RGD peptide mimetics, and anti-E-selectin Fab.

15. The method of claim 10, wherein the ligand binds a receptor selected from the group consisting of folate receptor, E-selectin receptor, L-selectin receptor, P-selectin receptor, CD4 receptor, αβ integrin receptors and chemokine receptors.

16. A pharmaceutical composition comprising:
a) a therapeutically effective amount of 7-t-butoxyiminomethylcamptothecin entrapped in liposomes; and
b) a pharmaceutically acceptable excipient.

17. The pharmaceutical composition according to claim 16 wherein the liposomes are made from synthetic or natural phospholipids that are selected from the group consisting of phosphatidylcholine, distearoylphosphatidylcholine, sphingomyelin, diacyl glycerol, phosphatidyl ethanolamine, phosphatidylglycerol, distearylphosphatidylcholine and distearyl phosphatidylethanolamine.

18. The pharmaceutical composition according to claim 16. wherein the liposomes are surface grafted with a hydrophilic polymer.

19. The pharmaceutical composition according to claim 18 wherein the hydrophilic polymer is selected from the group consisting of polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol and polyaspartamide.

20-21. (canceled)

22. A method for the treatment of disease symptoms that are caused by the activation of the topoisomerase I receptor, comprising administering an effective amount of a pharmaceutical composition according to claim 16 to a patient in need thereof.
